(19) United States
(12) Patent Application Publication
(10) Pub. No.: US 2005/0277801 A1
(43) Pub. Date: Dec. 15, 2005

(54) Combined process for recovering hydrogen, ethylene, ethane or separating ethylene cracked gas from dry gas of refinery plants

(76) Inventors: Guangjin Chen, Beijing (CN); Xuqiang Guo, Beijing (CN); Guanjing Wu, Beijing (CN); An Ma, Beijing (CN); Lijun Yan, Beijing (CN); Hu Luo, Beijing (CN); Changyu Sun, Beijing (CN)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 11/014,933

(22) Filed: Dec. 20, 2004

(30) Foreign Application Priority Data
Jun. 11, 2004 (CN) 200410047916.9

(51) Int. Cl. C07C 7/00

(52) U.S. Cl. 585/802; 585/820; 585/15

(57) ABSTRACT

This application provides processes for recovering hydrogen, ethylene and ethane from dry gas or separating ethylene cracked gas from dry gas of refinery plants, wherein a hydrating separation may be combined with for example a freezing or absorbing separation so as to separate a multiple gas mixture. The process may include for example: providing a dry gas or ethylene cracked gas to be separated by hydrating separation into a hydrating reactor to produce a hydrate comprising methane and nitrogen and a first gas mixture comprising hydrogen, ethane, and ethylene; removing the hydrate from the first gas phase mixture; further separating the first gas phase mixture of comprising hydrogen, ethylene and ethane in a conventional freezing or absorbing separation; and providing the hydrate produced in the hydration reactor into a hydrate decomposer wherein the hydrate is decomposed under the conditions of heating or decompression to obtain a second gas mixture comprising methane and nitrogen and less amount of ethane and ethylene.

### FIELD OF THE INVENTION

This invention relates to chemical engineering technology, particularly to a process for separating and recovering hydrogen, ethylene, ethane from the dry gas or separating ethylene cracked gas from dry gas of refinery plants in combination of hydrating separation technology with refrigerating separation or absorbing separation technology to separate and recover hydrogen, ethylene, ethane or separate ethylene cracked gas from the dry gas of refinery plants.

### TECHNICAL BACKGROUND OF THE INVENTION

A refinery plant generally produces a great deal of dry gases, such as catalytic cracked gas and splitting gas. These dry gases are complicated in composition, and their major components mainly include H₂, N₂, CH, C₂H₄, C₂H₆ and CO₂ and the like. These dry gases are gases mixture of low boiling point, wherein the components of H₂ and C₂ (such as C₂H₁ and C₂H₆) have high economic value, and the refinery enterprises, though having strong wishes to recover them, have not recovered them actually at present because the existing separation methods such as deep. freezing separation, absorption with tension variation, and film separation are not practical economically when they are applied in these systems.

As a cornerstone of petrochemical industry, the ethylene industry has been playing an important role in the national economy. Most of the existing ethylene works run under the pressure of capability expansion and profit promotion. The most complicated part of an ethylene device is the section of deep freezing for demethanation, which is the bottle neck in capability expansion and profit promotion for the whole device. The hydrating separation technology can exactly meet the requirements for separation of the above mentioned low boiling point gaseous mixture.

Hydrate is a kind of cage-type material formed up by water and small molecule gases (CH₄, C₂H₆, CO₂, N₂, etc.) under a certain temperature and pressure conditions, wherein water molecules build up cages that are connected together with hydrogen bonds, and gas molecules stay in the cages to maintain their stability. Different gases are at different levels of difficulty to form up hydrate, so it can be adopted to separate gases with the gases that are easy to form up hydrate entering into hydrate first. Because generally only small molecule gases can form up hydrate, the hydrating method can only be adaptable to low boiling point gas mixtures. It is more effective to use traditional rectification to separate the gases mixture of which the boiling point is not so low. The greatest advantage of hydrating method is that the low boiling point gases can be separated at the temperature above 0° C., while the traditional : rectification method requires very low temperature to do so, for example, with the latter method, methane and hydrogen should be separated at −160° C. while methane and ethane should be separated at −110° C.

### SUMARRY OF THE INVENTION

In order to overcome the disadvantage of the prior art, the object of present invention is to provide a process for recovering hydrogen, ethylene, ethane from dry gas or separating ethylene cracked gas from dry gas of refinery plants, wherein the hydrating separation is combined with the freezing or absorbing separation so as to separate multiple gases mixture, comprising the steps of:
(1) providing the dry gas or ethylene cracked gas to be separated into the hydrating separation, removing hydrate produced mainly by methane and nitrogen from gas phase, obtaining the remaining mixture mainly containing hydrogen, ethane, and ethylene in gas phase;
(2) separating the mixture of hydrogen, ethylene and ethane obtained in the hydrating separation further in conventional freezing or absorbing separation to obtain hydrogen, ethylene and ethane as product in two material streams for leaving from the separation system;
(3) providing the hydrate produced in hydration reactor during the hydrating separation into hydrate decomposer wherein the hydrate is decomposed under the conditions of heating or decompression to obtain gases mixture mainly containing methane and nitrogen and less amount of ethane and ethylene.
   wherein the hydrating separations include the procedures wherein the gases mixture reacts with water to generate hydrate and the hydrate decomposes and releases water and gases respectively in the hydrate reactor and hydrate decomposer, water is circulated between the hydrate reactor and hydrate decomposer.

According to the present invention, a selective thermodynamic accelerant is added into circulating water in the hydrating separation. The selective thermodynamic accelerant includes tetrahydrofuran, ethylene oxide, cyclopentane and acetone, preferably tetrahydrofuran. The said tetrahydrofuran is added as selective thermodynamic accelerant in water in amount of 5% to 15% in mol density.

In the present invention, a dynamic accelerant is also added into circulating water in the hydrating separation in amount of 500 mg/liter to 800 mg/liter in aqueous phase. The dynamic accelerant includes sodium lauryl sulphate (SDS), and sodium dodecyl benzene sulfonate (SDBS).

The present invention is also to provide a combined process for recovering hydrogen, ethylene, ethane from the dry gas or separate ethylene cracked gas of refinery plants, wherein the first and second hydrating separations are combined with the refrigerating or absorbing separation so as to separate multiple gases mixture, comprising the steps of:
(1) providing the dry gas or ethylene cracked gas to be separated into the first hydrating separation, removing hydrate produced mainly by methane and nitrogen from gas phase, obtaining the remaining mixture mainly containing hydrogen, ethane, and ethylene in gas phase;
(2) separating the mixture of hydrogen, ethylene and ethane obtained in the first hydrating separation further in conventional refrigerating or absorbing separation to obtain hydrogen, ethylene and ethane as product in two material streams for leaving from the separation system;
(3) providing the hydrate produced in hydration reactor during the first hydrating separation into hydrate decomposer wherein the hydrate is decomposed under the conditions of heating or decompression to obtain gases mixture mainly containing methane and nitrogen and less amount of ethane and ethylene;
(4) providing the gases mixture obtained in the hydrate decomposer during the first hydrating separation into the second hydrating separation wherein ethane and ethylene preferably turn into hydrates, and the remaining gas phase that mainly contains methane or the mixture of methane and nitrogen as out-going material stream leaves the separation system;
(5) returning a gas phase formed in the decomposition of hydrate generated in the second hydrating separation into the hydrate reactor in the first hydrating separation as circulating material.

According the invention, both the first and second hydrating separations include the procedures wherein the gases mixture reacts with water to generate hydrate and the hydrate decomposes and releases water and gases respectively in the hydrate reactor and hydrate decomposer, water is circulated between the hydrate reactor and hydrate decomposer.

A selective thermodynamic accelerant is added into the circulating water in the first hydrating separation. Such selective thermodynamic accelerant includes tetrahydrofuran, ethylene oxide, cyclopentane and acetone, preferably tetrahydrofuran. The said tetrahydrofuran is added as selective thermodynamic accelerant in water in amount of 5% to 15% in mol density.

According to the invention, dynamic accelerants are also added into the circulating water both in the first hydrating separation and the second hydrating separation in amount of 500 mg/liter to 800 mg/liter in aqueous phase. The said dynamic accelerants include sodium lauryl sulphate (SDS), and sodium dodecyl benzene sulfonate (SDBS).

In addition, the absorbents are also used in the absorbing separation and include light oil, methyl alcohol and tetrahydrofuran; the operating temperature ranges from −30° C. to 0° C., the pressure is 1˜3 Mpa during the separation.

The advantages of the invention are given below:
(1) The present invention provides a process for separating and recovering hydrogen, ethylene, ethane or separating ethylene cracked gas from the dry gas of refinery plants. For these gases mixture, the conventional rectifying methods require very low temperature (below −100° C.), while other methods, such as membrane separation, absorption with tension variation are neither applicable to some gases which this invention involves, such as the gases mixture with complicated components like catalytic dry gas, cracked dry gas, and ethylene cracked gas, because of difficulties in de-absorption, high consumption of the membrane and low separation efficiency.
(2) At the same time, it is likely that the hydrating method will gain competitive advantages in equipment investment and operating cost, and reduce operating difficulties.

The invention having the combination of processes is applicable to the following two aspects:
(1) Recovery of hydrogen from refinery dry gases (catalytic dry gas ethylbenzene and etc.) that are produced in high rate. These dry gases are in high production rate and complicated in components with hydrogen density ranging from 15%-40%. The process of the present invention can recover and concentrate hydrogen, and recover C₂ components that are high in economic value.
(2) Application in ethylene production process. It can be used to remove most of the methane from cracked gas before deep freezing so as to lower the cold load in deep freezing.

### BRIEF DESCRIPTION OF THE DRAWINGS

FIG. 1 is the illustration of process of the present invention. FIG. 2 is the diagram of system pressure varying against time with different SDS density (T=281.65K).

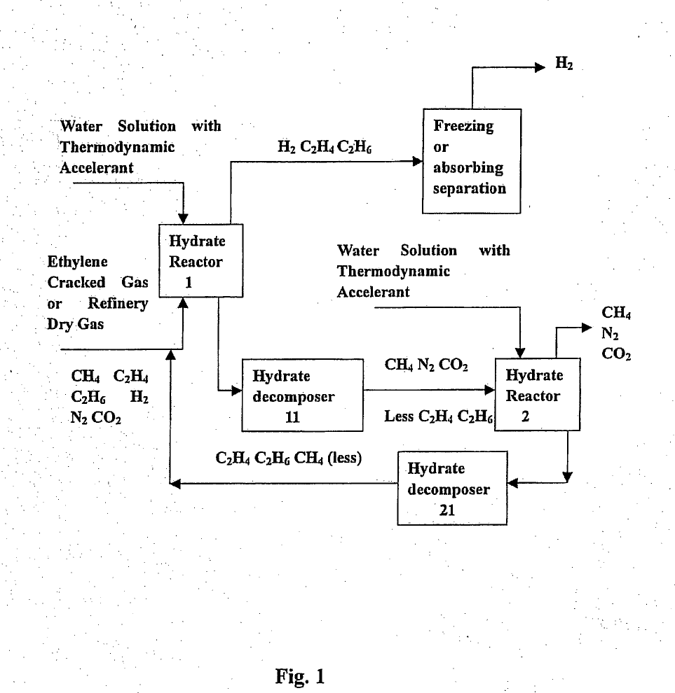

FIG. 3 is a comparison between a pure water system and a 500 ppm SDS system ethylene hydrate generation process.

### DETAILED DESCRIPTION OF THE INVENTION

The invention is to provide a group of processes used to recover hydrogen, ethylene, ethane or separate ethylene cracked gas from the dry gas of refinery plants, and promote the efficiency to extract the gases of high economic value from the dry gas or cracked ethylene gas of refinery plants and save energy for a further step.

At first, first hydrating separation includes that before entering the tower type hydrating reactor, the raw gases are pre-pressurized and pre-cooled; the pressure is not lower than 5 Mpa and the cooling temperature is 1-4° C. Then the raw gases enter the reactor from its bottom, and the gases in their upward movement contact continuously with the downward moving water solution containing thermodynamic accelerant and dynamic accelerant and they generate. hydrate. Because a selective thermodynamic accelerant such as tetrahydrofuran is added into hydrating reactor 11 to lower the generating pressure of hydrate, and at the same time, tetrahydrofuran can also occupy the big apertures in hydrate lattice so as to dramatically suppress the bigger molecules like those of ethane or ethylene to form hydrate and sufficiently separate methane and ethylene as well as ethane from each other. A dynamic accelerant is also added into the water solution to promote hydrate's generating speed and prevent the system from being jammed.

Two material streams are obtained in hydrating reactor I, one of the streams is the gaseous stream mainly containing hydrogen, ethane and ethylene led out from the top of the reactor and it enters the freezing or absorption separation system to separate hydrogen and C₂ components (ethane and ethylene). The other stream is a kind of solution formed up by hydrates and the water solution that has not taken part in reaction. This stream enters hydrating reactor I for decomposition to release gases and water solution. The water solution after decomposition returns to the top of the reactor after cooling for circulating use. A little amount of C₂ components are contained in the gases released from decomposition and should be recovered further.

The second hydrating separation includes that the gases coming from hydrating decomposer II are let into hydrating reactor tower 2. During up-going, the gases enter tower-type hydrating reactor 21 from its bottom and keep contacting stage by stage reversibly with the downward moving water solution containing dynamic accelerant to generate hydrate. With different pressure for different gases to generate hydrate, C₂ components in small amount in the gases are separated from the other gases. The components that are easy to generate hydrate (C₂ components) are turned into hydrate and are mixed up with the water solution to turn into solid mixtures. The remaining gases (CH₄, N₂, CO₂) are exhausted from the top of hydrating reactor I and leave from the separation system. The solid mixture is sent to hydrating decomposer II to decompose into water solution and gases mixture with relatively high contents of C₂ components. The gases mixture is pressurized and sent back to the bottom of hydrating reactor I to recover C₂ components. And then the water solution is sent back to hydrating reactor II for circulating use after being cooled.

If freezing separation method is adopted to separate the mixture of hydrogen and C₂, then the gases (the mixture of hydrogen, ethylene and ethane) that come from the top 5. of tower hydrating reactor I and have not turned into hydrate should be frozen to −10˜−20° C. with an external freezer to get separated liquid and gases, they should be further frozen to lower their temperature, then be throttled and sent into the freezing separation device to get hydrogen and C₂ components.

With absorption separation adopted, the gases (the mixture of hydrogen, ethylene and ethane) that come from the top of tower-type hydrating reactor and have not turned into hydrate are directly sent into the absorbing separation device, and hydrogen and C₂ components can be obtained after absorption and de-absorption.

In addition, a dynamic accelerant can be added in the circulating water solution in both the said first and second hydrating separations. That is to say, the dynamic accelerant can be added into the water solution to accelerate hydrate. generating speed and suppress hydrate grains to mass and jam the system in the first separation process and the second separation process.

The first hydrating separation is carried out by adding the selective thermodynamic accelerant into circulating: water solution to promote the formation of hydrate of ethane and nitrogen and the like and to suppress the formation of hydrate of components of target product such as hydrogen, ethylene as well as ethane in order to increase efficiency of the separation and decrease the operating pressure.

The thermodynamic accelerant makes it easier for gases to turn into hydrate. Table 1 shows the data of pure methane hydrate generation conditions in the water solution of 6% (mol percentage) tetrahydroflran, while. Table 2 supplies equilibrium data of methane hydrate generation in pure water.

TABLE 1
Pressure and Temperature for CH₄ Hydrate Generation
with Tetrahydrofuran Added

| | | | | | |
|---|---|---|---|---|---|
| Temperature | 15.0 | 12.5 | 9.4 | 7.0 | 4.5 |
| ° C. | | | | | |
| Pressure MPa | 1.20 | 0.72 | 0.38 | 0.21 | 0.10 |

TABLE 2
Pressure and Temperature for CH₄ Hydrate Generation
without Accelerant Added

| | | | | | | | | | |
|---|---|---|---|---|---|---|---|---|---|
| Tem- | 17.6 | 15.4 | 12.3 | 10.9 | 9.7 | 8.2 | 7.3 | 5.0 | 4.0 |
| perature | | | | | | | | | |
| ° C. | | | | | | | | | |
| Pressure | 16.96 | 13.04 | 9.19 | 8.05 | 7.04 | 6.04 | 5.35 | 4.50 | 3.90 |
| MPa | | | | | | | | | |

Table 1 and Table 2 indicate that the pressure for generation of methane hydrate is dramatically decreased with addition of thermodynamic accelerant.

The selective thermodynamic accelerant used in the first hydrating separation is avoided to be used in the second hydrating separation so as to ensure that ethane and ethylene turn into hydrate prior to nitrogen.

Dynamic Accelerant

Promotion Effect of Dynamic Accelerant on Methane Hydrate Generation:

FIG. 2 shows methane hydrate generating speeds in solutions of different levels of SDS density and in pure water. The figure shows that, with addition of surface-active agent SDS, hydrate-generating speed in the first ten minutes is much faster than the case without surface-active agent. And in the first ten minutes, pressure variation exceeds 0.5 MPa with addition of surface-active while only reaches 0.25 Mpa without addition of surface-active. The preferable effect is achieved at 400 ppm, it drops over 0.65 Mpa within the first ten minutes.

Promotion Effect of SDS on Ethylene Hydrate Generation:

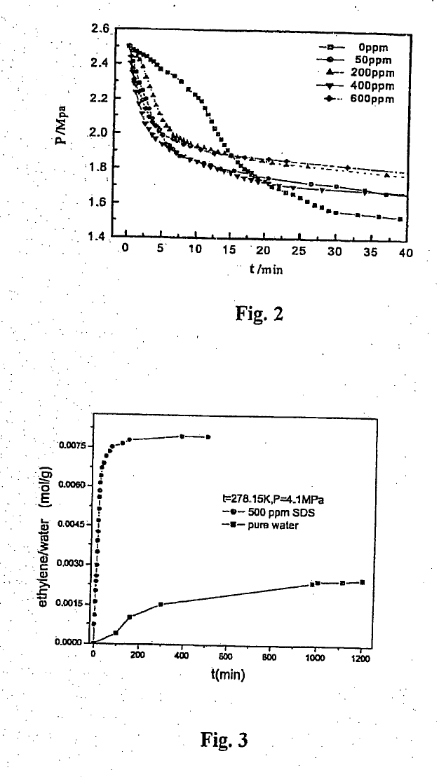

As is shown in FIG. 3, under the conditions of t-278.15 and p=4.1 Mpa, hydrate generation speed in static pure water system is slow, and converting period takes a long period with a very low rate for each gram of water converting into hydrate as a final result. On the contrary, the generating speed of ethylene hydrate in SDS solution system is much faster and the ethylene quantity finally converted into hydrate is also much higher than the quantity in pure water system.

### EXAMPLE 1

### Single-Stage Separation of Methane and Ethane

Gases mixture with methane and ethane was prepared in laboratory, the gases was put in a stirring volume-variable reactor containing tetrahydrofuran (TFT) water solution to generate hydrate. When the reaction reaches balance, the composition of gaseous components in gas phase was analyzed and was sampled for analysis of decomposition components. The results as shown below:

TABLE 3
Phase Balance Constants of Hydrate Generated with
CH₄(1) + C₂H₆(2) Mixed Gases
When Selective Suppressor Added
(THF density = 6%, Z₂ = 6% mol)

| | Reaction pressure | | | | | | |
|---|---|---|---|---|---|---|---|
| | P = 2.0 MPa | | | | P = 3.0 Mpa | | |
| | Reaction temperature | | | | | | |
| | 3.0 | 5.0 | 7.0 | 9.0 | 5.0 | 7.0 | 9.0 |
| Y2% | 73.73 | 87.68 | 85.49 | 80.33 | 79.71 | 87.08 | 80.74 |
| X2% | 28.89 | 27.80 | 21.37 | 14.74 | 25.46 | 23.44 | 19.55 |
| K2% | 2.55 | 3.15 | 4.00 | 5.45 | 3.13 | 3.72 | 4.13 |

Note:
In the table, y1 and x1 respectively stand for ethane mol percentage in gas phase and ethane mol percentage in hydrate.

TABLE 4
Phase Balance Constants of Hydrate Generated with CH₄(1) + C₂H₆(2)
Mixed Gases When No Selective Suppressor Added
(Z2 = 60.11%)

| T(° C.) | P(MPa) | X₂(mol %) | Y₂(mol %) | K₂ |
|---|---|---|---|---|
| 1.0 | 2.5 | 65.92 | 58.25 | 0.88 |
| | 3.0 | 65.67 | 57.73 | 0.88 |
| | 4.0 | 62.53 | 57.84 | 0.92 |

As is shown in the tables, because selective suppressor-accelerant THF is used in the process of hydrating separation, the phase balance constants of ethane are reversed, and the maximum density difference between two phases reaches 64%. Higher THF density is favorable in suppressing ethane to turn into hydrate so as to lower the content of ethane in the phase of hydrate and promote the distributing coefficient of ethane. in two phases.

### EXAMPLE 2

### Single-Stage Separation of Methane and Ethylene

Gases mixture with methane and ethane was prepared in laboratory, the gases was put in a stirring reactor of fixed volume containing TFT water solution to generate hydrate.

When the reaction reaches balance, the composition of gaseous components in gas phase was analyzed and sampled for analysis of decomposition components. The results of the experiments are as shown in Table 5.

TABLE 5
Experimental Results of Ethane and Ethylene Single-stage Separation

| | Reaction | Initial | Balanced | Reaction | Initial | THF | | |
|---|---|---|---|---|---|---|---|---|
| | temperature | pressure | pressure | time | gas-liquid | density | | |
| Z₁(%) | (° C.) | (MPa) | (MPa) | (min.) | Ratio | (mol %) | Y₁ (%) | X₁ (%) |
| 58.69 | 5 | 2.0 | 1.30 | 11 | 40:1 | 6.0 | 75.42 | 44.09 |
| 58.69 | 5 | 2.5 | 1.55 | 15 | 56:1 | 6.0 | 75.76 | 31.17 |
| 76.50 | 7 | 2.0 | 1.40 | 25 | 60:1 | 6.0 | 89.64 | 56.48 |
| 76.50 | 5 | 2.0 | 1.15 | 30 | 40:1 | 6.0 | 91.35 | 56.10 |
| 76.50 | 5 | 2.5 | 1.75 | 20 | 40:1 | 6.0 | 92.60 | 58.91 |
| 78.82 | 5 | 2.0 | 1.15 | 30 | 60:1 | 8.0 | 95.68 | 67.47 |
| 78.82 | 5 | 2.0 | 1.35 | 20 | 40:1 | 8.0 | 91.40 | 58.48 |
| 75.75 | 5 | 2.0 | 1.40 | 9 | 40:1 | 8.0 | 90.03 | 59.96 |
| 90.66 | 5 | 2.0 | 1.10 | 33 | 40:1 | 6.0 | 96.82 | 87.49 |
| 90.66 | 5 | 2.0 | 1.09 | 40 | 40:1 | 8.0 | 97.33 | 87.53 |
| 90.66 | 5 | 2.0 | 1.20 | 30 | 40:1 | 10.0 | 97.38 | 81.36 |
| 90.66 | 5 | 2.0 | 1.00 | 38 | 40:1 | 12.0 | 96.31 | 88.21 |

Note:
In the table, Z₁, Y₁ and X₁ respectively stand for mol percentage of ethylene in the inlet gas, mol percentage of ethylene in gas phase, and mol percentage of ethylene in hydrate phase.

From above table it indicates that ethylene density in gas phase is dramatically increased after one -stage separation of hydrate.

### EXAMPLE 3

### Separation of Multiple-Element Mixed Gases

In light of gas separation in ethylene production and recovery of hydrogen and C₂ components from refinery dry gas, experiments were carried out under different conditions for hydrate single-stage one-time separation, with the experimental results listed in Table 6. The table indicates that compared to the raw gas, methane density in gas phase is dramatically decreased, while the major element in solid phase (hydrate phase) is methane. This shows that hydrating method can remove methane in gas phase obviously and recover and condense the components with high economic value like hydrogen and C₂.

TABLE 6
One-time Separation Experimental Data of H₂—C₂H₄—C₂H₆—CH₄ Mixture System
(Inlet material components: 33.963% H₂ + 16.564% C₂H₄ + 16.7% C₂H₄ + 32.773%
CH₄)

| | | | | | | | | | | Solid dry basis | | | |
|---|---|---|---|---|---|---|---|---|---|---|---|---|---|
| Reaction | Initial | Balance | Reaction | Initial gas- | THF | Gas composition (%) | | | | composition (%) | | | |
| temperature (° C.) | pressure (MPa) | pressure (MPa) | time (min) | liquid ratio | density (%) | H₂ | C₂H₄ | C₂H₆ | CH₄ | H₂ | C₂H₄ | C₂H₆ | CH₄ |
| 5.0 | 5.0 | 3.97 | 20 | 100:1 | 6.0 | 51.7 | 16.0 | 22.0 | 10.3 | 0.53 | 14.8 | 7.77 | 77.8 |
| 5.0 | 5.0 | 3.92 | 6 | 40:1 | 6.0 | 54.8 | 13.4 | 23.7 | 7.96 | 7.22 | 18.1 | 10.0 | 64.5 |
| 5.0 | 4.0 | 3.00 | 10 | 50:1 | 6.0 | 50.6 | 14.9 | 22.6 | 11.8 | 5.58 | 17.5 | 7.40 | 69.5 |
| 5.0 | 4.0 | 3.02 | 10 | 60:1 | 6.0 | 51.6 | 15.5 | 23.7 | 9.38 | 7.78 | 17.1 | 7.97 | 67.1 |
| 5.0 | 3.0 | 2.49 | 10 | 40:1 | 10.0 | 49.2 | 15.2 | 21.9 | 13.6 | 6.13 | 17.5 | 7.63 | 68.7 |

### EXAMPLE 4

### Multi-Stage Separation Results of Hydrate

In order to examine multi-stage separation effects, twice-separation experiments were conducted with the product in gas phase listed in Table 4 with the hydrating method and the results are as shown in Table 5. Table 5 shows that methane in gas phase is further decreased dramatically with time times of separation while the ratio of C₂ and methane in gas phase is increased, which indicates C₂ has been further condensed (if hydrogen is deducted).

TABLE 7
Two-time Separation Experimental Data of H2—C2H4—C2H6—CH4 Mixture System

| | | | | | | | | | | Solid dry basis | | | |
|---|---|---|---|---|---|---|---|---|---|---|---|---|---|
| Reaction tem- | Initial | Balance | Reaction | Initial gas- | THF | Gas composition (%) | | | | composition (%) | | | |
| perature (° C.) | pressure (MPa) | pressure (MPa) | time (min.) | liquid ratio | density (%) | H₂ | C₂H₄ | C₂H₆ | CH₄ | H₂ | C₂H₄ | C₂H₆ | CH₄ |
| 5 | 5.0 | 4.47 | 10 | 40:1 | 6.0 | 58.4 | 9.75 | 30.3 | 1.51 | 27.9 | 23.4 | 22.5 | 26.2 |
| 5 | 4.0 | 3.53 | 10 | 40:1 | 6.0 | 57.8 | 10.8 | 28.4 | 3.02 | 25.1 | 23.6 | 22.3 | 29.0 |
| 5 | 5.0 | 4.44 | 10 | 40:1 | 8.0 | 58.3 | 10.5 | 30.1 | 1.09 | 34.3 | 0.14 | 0.28 | 65.3 |
| 5 | 5.0 | 4.44 | 10 | 40:1 | 10.0 | 56.1 | 11.5 | 29.5 | 2.96 | 15.3 | 26.6 | 18.6 | 39.5 |
| 5 | 5.0 | 4.55 | 10 | 40:1 | 12.0 | 57.3 | 11.9 | 27.7 | 3.01 | 45.1 | 15.0 | 28.3 | 11.6 |

Although the present invention has been described hereinabove by way of preferred embodiment thereof, it can be modified without departing from the spirit and nature of the subject invention as defined in the appended claims.

1. A process for recovering hydrogen, ethylene, and. ethane from dry gas or separating ethylene cracked gas from dry gas of refinery plants, wherein a hydrating separation is combined with a freezing or absorbing separation so as to separate a multiple gases mixture, comprising the steps of:
(1) providing a dry gas or ethylene cracked gas to be separated by hydrating separation into a hydrating; reactor,
(2) removing the hydrate produced mainly by methane and nitrogen from the gas phase, thereby obtaining the remaining mixture mainly comprising hydrogen, ethane, and ethylene in the gas phase;
(3) further separating the mixture comprising hydrogen, ethylene and ethane obtained in the hydrating separation in a conventional freezing or absorbing separation to obtain hydrogen, ethylene and ethane as a product in two material streams for leaving the separation system; and
(4) providing the hydrate produced in said hydration reactor during the hydrating separation into a hydrate decomposer wherein the hydrate is decomposed under the conditions of heating or decompression to obtain a gases mixture mainly comprising methane and nitrogen and less amount of ethane and ethylene.

2. The process of claim 1, wherein the hydrating separation comprises the step of reacting the gases mixture with water to generate a hydrate, wherein the hydrate decomposes and releases water and gases respectively in the hydrate reactor and the hydrate decomposer, wherein water is circulated between the hydrate reactor and hydrate decomposer.

3. The process of claim 2, wherein a selective thermodynamic accelerant is added into the circulating water in the hydrating separation.

4. The process of claim 3, wherein the selective thermodynamic accelerant comprises tetrahydrofuran, ethylene oxide, cyclopentane, acetone or any combination thereof.

5. The process of claim 3, wherein the selective thermodynamic accelerant is tetrahydrofuran.

6. The process of claim 5, wherein said tetrahydrofuran is added to the water in an amount of 5% to 15% in mol density.

7. The process of claim 3, wherein said thermodynamic accelerant is added into circulating water in the hydrating separation in amount of 500 mg/liter to 800 mg/liter in aqueous phase.

8. The process of claim 7, wherein the thermodynamic accelerant comprises sodium lauryl sulphate (SDS), sodium dodecyl benzene sulfonate (SDBS) or any combination thereof.

9. A combined process for recovering hydrogen, ethylene and ethane from dry gas or separating ethylene cracked gas from dry gas of refinery plants, wherein a first and a second hydrating separations are combined with a freezing or absorbing separation so as to separate a multiple gases mixture, comprising the steps of:
(1) providing a dry gas or ethylene cracked gas to be separated by a first hydrating separation into a first hydrating reactor,
(2) removing the hydrate produced mainly by methane and nitrogen from the gas phase, thereby obtaining the remaining mixture mainly containing hydrogen, ethane, and ethylene in the gas phase;
(3) further separating the mixture comprising hydrogen, ethylene and ethane obtained in the first hydrating separation in a conventional freezing or absorbing separation to obtain hydrogen, ethylene and ethane as a product in two material streams for leaving the separation system;
(4) providing the hydrate produced in said first hydration reactor during the first hydrating separation into a hydrate decomposer wherein the hydrate is decomposed under the conditions of heating or decompression to obtain a gases mixture mainly comprising methane and nitrogen and less amount of ethane and ethylene,
(5) providing the gases mixture obtained in the hydrate decomposer during the first hydrating separation into a second hydrating reactor for a second hydrating separations wherein ethane and ethylene preferably turn into hydrates, and the remaining gas phase that mainly comprises methane or a mixture of methane and nitrogen as out-going material stream leaves the separation system; and
(6) returning a gas phase the gases mixture formed in the decomposition of the hydrate generated during the second hydrating separation into the first hydrating reactor as circulating material.

10. The process of claim 9, wherein both first and second hydrating separations comprise the step of reacting the gases mixture with water to generate a hydrate, wherein the hydrate decomposes and releases water and gases respectively in the hydrate. reactor and the hydrate decomposer and wherein water is circulated between the hydrate reactor and hydrate decomposer.

11. The process of claim 9, wherein a selective thermodynamic accelerant is added into the circulating water in the first hydrating separation.

12. The process of claim 11, wherein the selective thermodynamic accelerant comprises tetrahydrofuran, ethylene oxide, cyclopentane, acetone or any combination thereof.

13. The process of claim 11, wherein the selective thermodynamic accelerant is tetrahydrofuran.

14. The process of claim 13, wherein the tetrahydrofuran is added to the water in an amount of 5% to 15% in mol density.

15. The process of claim 11, wherein the thermodynamic accelerant is added into the circulating water both in the first hydrating separation and the second hydrating separation in an amount of 500 mg/liter to 800 mg/liter in aqueous phase.

16. The process of claim 15, wherein the thermodynamic accelerant includes comorises sodium lauryl sulphate (SDS), sodium dodecyl benzene sulfonate (SDBS) or any combination thereof.

17. The process of claim 9, wherein an absorbant absorbents is used in the absorbing separation, wherein said absorbent comprises light oil, methyl alcohol, tetrahydrofuran or any combination thereof.

18. The process of claim 9, wherein the operating temperature in said absorbing separation ranges from −30° C. to 0° C.

19. The process of claim 9, wherein the pressure during said absorbing separation is 1˜3 Mpa.
